(19) United States
(12) Patent Application Publication
(10) Pub. No.: US 2026/0306256 A1
(43) Pub. Date: Oct. 8, 2026

(54) PROSTHETIC VALVES WITH SKIRT SUPPORT FEATURES

(71) Applicant: Edwards Lifesciences Corporation, Irvine, CA (US)

(72) Inventors: Tamir S. Levi, Zikhron Yaakov (IL); Gal Pereg, Tel-Aviv (IL); Anatoly Dvorsky, Haifa (IL); Elena Sherman, Pardes Hana (IL)

(21) Appl. No.: 19/665,928

(22) Filed: May 1, 2026

Related U.S. Application Data
(63) Continuation of application No. PCT/US2024/055068, filed on Nov. 8, 2024.
(60) Provisional application No. 63/598,057, filed on Nov. 10, 2023.

(51) Int. Cl. A61F 2/24

(52) U.S. Cl. CPC A61F 2/2418; A61F 2/2433; A61F 2250/0018; A61F 2250/0036; A61F 2250/0051

(57) ABSTRACT

The present disclosure relates to prosthetic valves equipped with elongated skirts extending distally to the frame. In an example, a prosthetic valve can include a plastically-expandable frame movable between a radially compressed state and a radially expanded state, a plurality of plastically deformable inflow prongs terminating at free ends positioned distal to an inflow end of the frame in the expanded state, and an outer skirt comprising a first portion disposed around the frame, and a second portion extending distal to the frame and disposed around the inflow prongs, wherein the inflow prongs are configured to limit a radially-inward oriented bending of the second portion of the outer skirt supported by the inflow prongs.

### CROSS-REFERENCE TO RELATED APPLICATIONS

This application is a continuation of PCT Patent Application No. PCT/US2024/055068, filed on Nov 8, 2024, which claims the benefit of U.S. Provisional Application No. 63/598,057, filed Nov 10, 2023, the entire disclosures of each of which being incorporated by reference herein.

### FIELD

The present invention relates to prosthetic valves that include elongated skirts extendable distal to the frames of the valve in expanded states thereof, and to features configured to limit inwardly-oriented bending of the skirt.

### BACKGROUND

Native heart valves, such as the aortic, pulmonary and mitral valves, function to assure adequate directional flow from, and to, the heart, and between the heart's chambers, to supply blood to the whole cardiovascular system. Various valvular diseases can render the valves ineffective and require replacement with artificial valves. Surgical procedures can be performed to repair or replace a heart valve. Conventional surgically implantable prosthetic valve can include a leaflet assembly mounted within a relatively rigid support frame or ring. Components of the prosthetic valve are usually assembled with one or more biocompatible fabrics, and a fabric-covered sewing ring is provided around the valve for suturing to the tissue of the native leaflet.

Since surgeries are prone to an abundance of clinical complications, alternative less invasive techniques of delivering a prosthetic heart valve over a catheter and implanting it over the native malfunctioning valve have been developed over the years. Different types of prosthetic heart valves are known to date, including balloon expandable valve, self-expandable valves and mechanically-expandable valves.

Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter.

### SUMMARY

Most expandable prosthetic valves include flexible leaflets attached to expandable frames thereof, wherein the leaflets are configured to transition between closed and open states, so as to regulate flow of blood through the prosthetic valves. Outer skirts covering the prosthetic valve are, in many cases, designed to encourage tissue overgrowth on their outer surface, so as to reduce risk of paravalvular leakage (PVL). When the inflow end of such a skirt is in close proximity to a native anatomical wall, such as that of the annulus in which the prosthetic valve is implanted, tissue growth may occasionally extend all the way to the inflow end of the skirt, and develop further along the inner side of the valve. Indeed, growths of tissue and pannus have been observed on leaflets in implanted prosthetic heart valves, particularly when an inflow end of the prosthetic valve is closer to the native annulus. Accordingly, a need exists for prosthetic valves that reduce the risk of tissue growth and pannus formation along the inner side of the valve and the leaflets.

According to some aspects of the disclosure, there is provided a prosthetic valve comprising a plastically-expandable frame extending between an inflow end and an outflow end, a plurality of plastically deformable inflow prongs attached to the frame and terminating at free ends positioned distal to the inflow end of the frame in the expanded state; and an outer skirt comprising a first portion disposed around the frame, and a second portion extending distal to the frame and disposed around the inflow prongs. The frame is movable between a radially compressed state and a radially expanded state.

In some examples, the inflow prongs are configured to limit a radially-inward oriented bending of the second portion of the outer skirt supported by the inflow prongs.

In some examples, the inflow prongs are configured to maintain an unbent configuration in a free state thereof.

In some examples, the inflow prongs are configured to bend radially outwards upon inflation of a balloon over which the prosthetic valve is crimped.

In some examples, the outer skirt is configured to assume an outwardly flared configuration in the expanded state.

In some examples, the free ends of the inflow prongs define a distal-most end of the inflow prongs in the expanded state.

In some examples, the first portion of the outer skirt comprises a textured surface configured to encourage tissue growth thereover.

In some examples, the second portion of the outer skirt is devoid of textured surfaces that may encourage tissue growth thereover.

In some examples, the second portion of the outer skirt is folded around the free ends of the inflow prongs.

In some examples, a fold of the outer skirt around the inflow prongs defines an inflow end of the prosthetic valve, which is distal to the inflow end of the frame in the expanded state.

In some examples, the prosthetic valve further comprises a valvular structure mounted inside the frame and comprising a plurality of leaflets.

According to some aspects of the disclosure, there is provided a prosthetic valve comprising a frame extending between an inflow end and an outflow end, an outer skirt comprising a first portion disposed around the frame and a second portion extending distal to the inflow end of the frame; and a reinforcing layer disposed over the second portion of the outer skirt. The frame is movable between a radially compressed state and a radially expanded state.

In some examples, the reinforcing layer is configured to limit a radially-inward oriented bending of the second portion of the outer skirt.

In some examples, the reinforcing layer is not disposed over the first layer of the outer skirt.

In some examples, a thickness of the second portion of the outer skirt, combined with the reinforcing layer, is greater than a thickness of the first portion of the outer skirt.

In some examples, an outer diameter of the prosthetic valve defined at a level of the second portion of the outer skirt is not greater than an outer diameter of the prosthetic valve defined at a level of the first portion of the outer skirt, in the crimped state.

In some examples, the reinforcing layer is stiffer than the outer skirt.

In some examples, the first portion of the outer skirt comprises a textured surface configured to encourage tissue growth thereover.

In some examples, the second portion of the outer skirt is devoid of textured surfaces that may encourage tissue growth thereover.

In some examples, the second portion of the outer skirt is folded over itself.

In some examples, a fold of the second portion of the outer skirt defines an inflow end of the prosthetic valve, which is distal to the inflow end of the frame in the expanded state.

In some examples, the prosthetic valve further comprises a valvular structure mounted inside the frame and comprising a plurality of leaflets.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

### In the Figures:

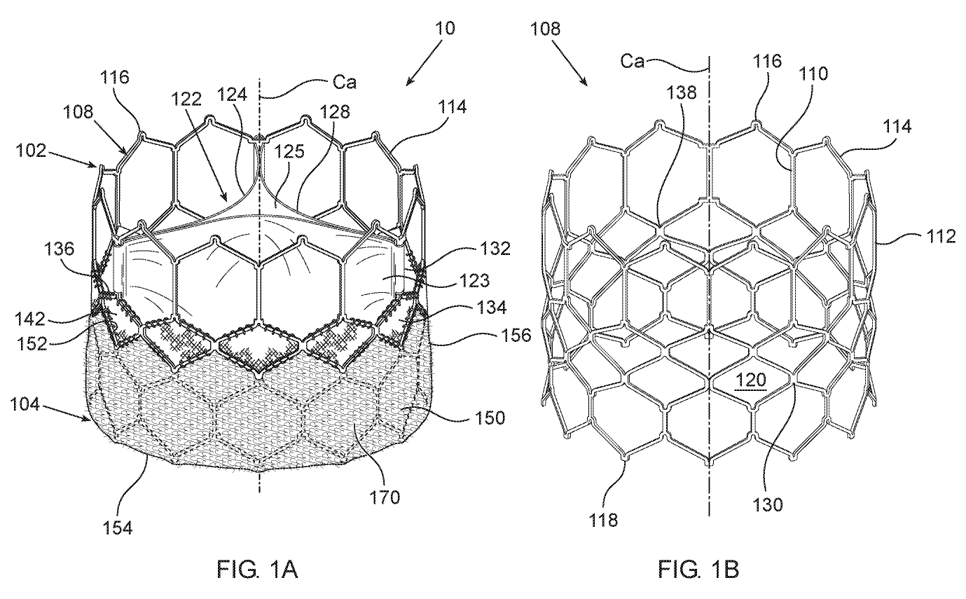

FIG. 1A is a perspective view of an exemplary prosthetic valve.

FIG. 1B is a perspective view of a frame of the prosthetic valve of FIG. 1A.

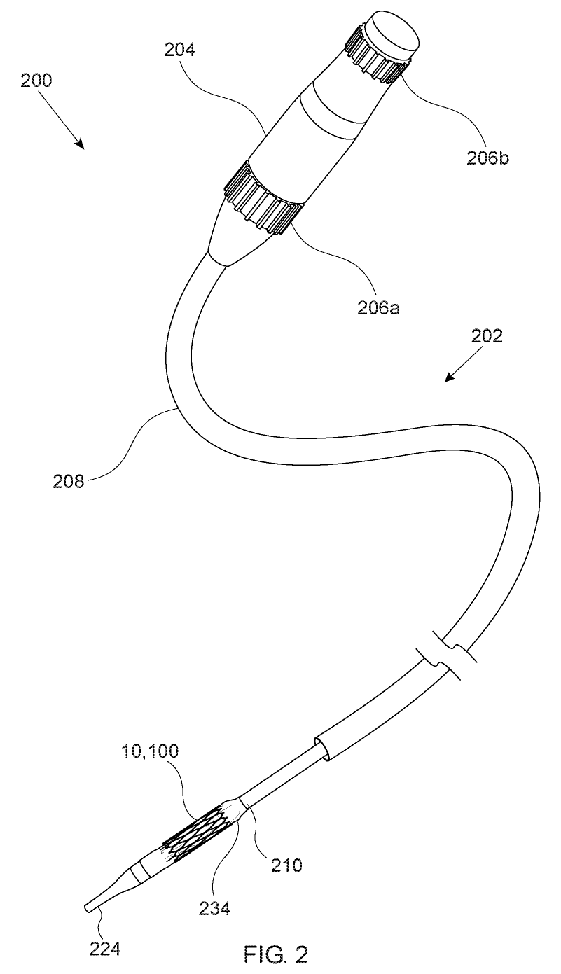

FIG. 2 shows an exemplary delivery apparatus carrying an exemplary balloon expandable prosthetic valve.

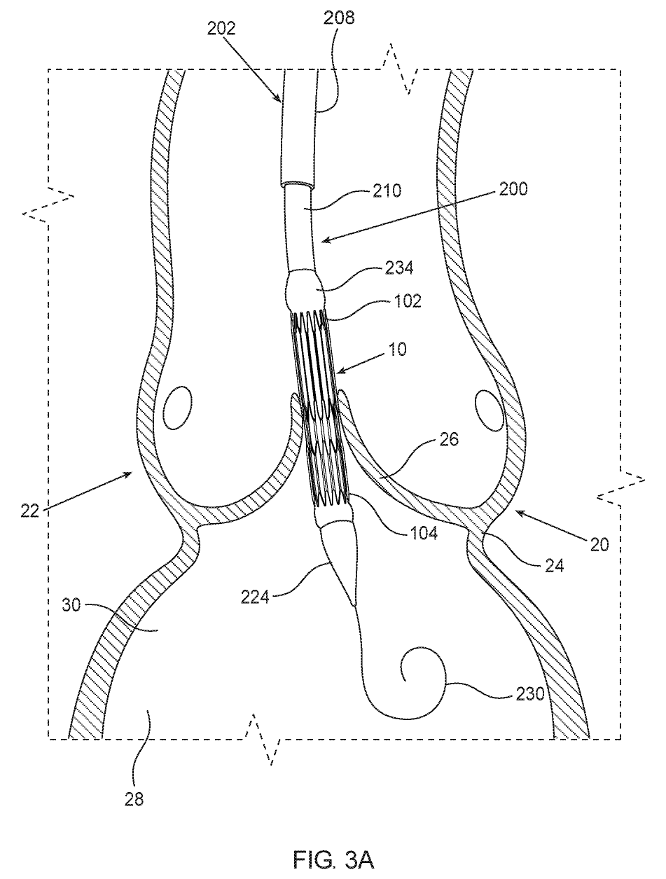

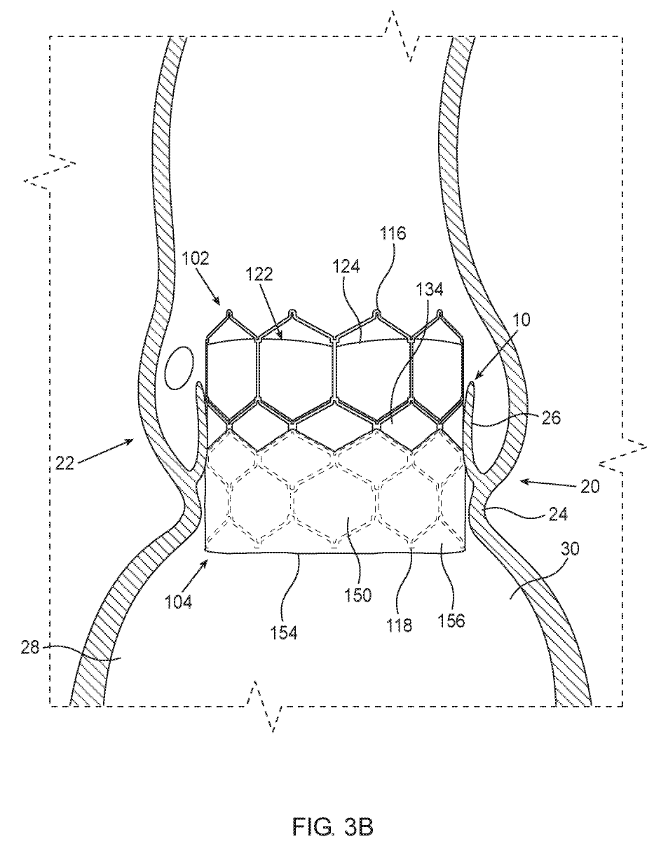

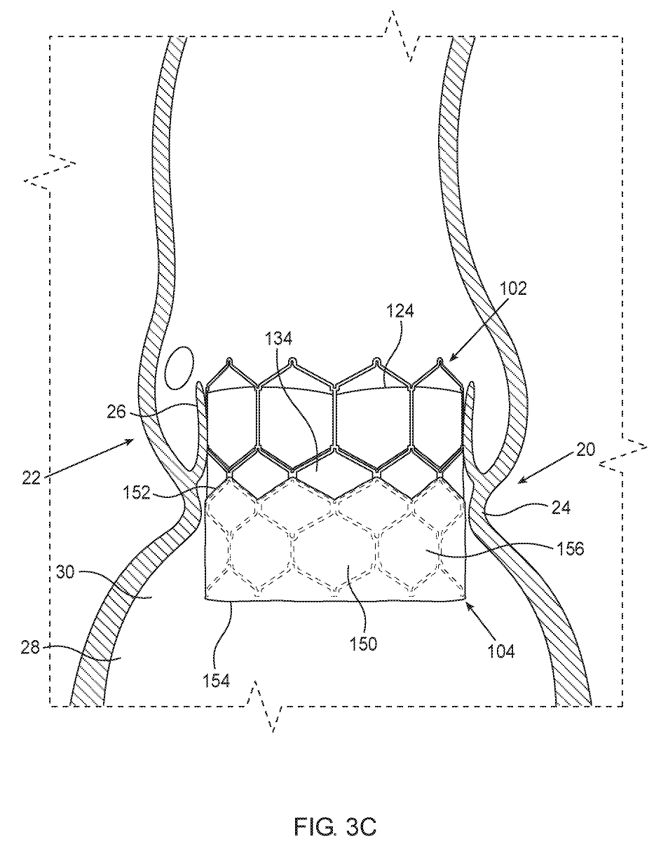

FIG. 3A shows a distal portion of a delivery assembly advanced to a native heat valve for implantation of a prosthetic valve therein.

FIGS. 3B and 3C show optional positions of a prosthetic valve implanted inside the native valve.

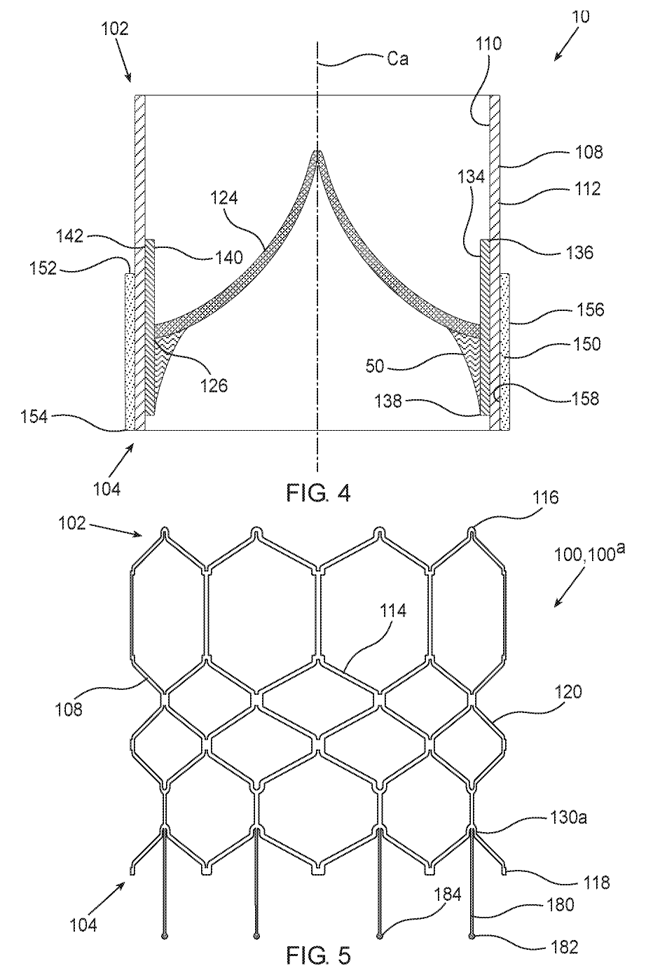

FIG. 4 is a cross-sectional view of an exemplary prosthetic valve, showing tissue overgrowth developed over the inner side of the prosthetic valve.

FIG. 5 shows a portion of a frame of an exemplary prosthetic valve, equipped with inflow prongs extending distally therefrom.

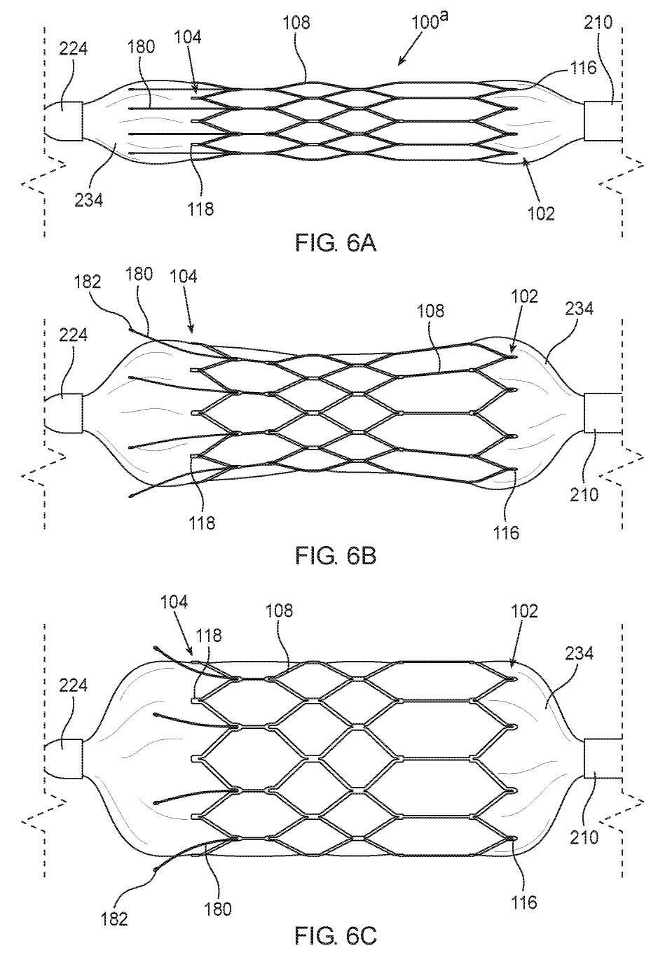

FIGS. 6A-6C illustrate optional phases in expansion of the prosthetic valve of FIG. 5 using an inflatable balloon.

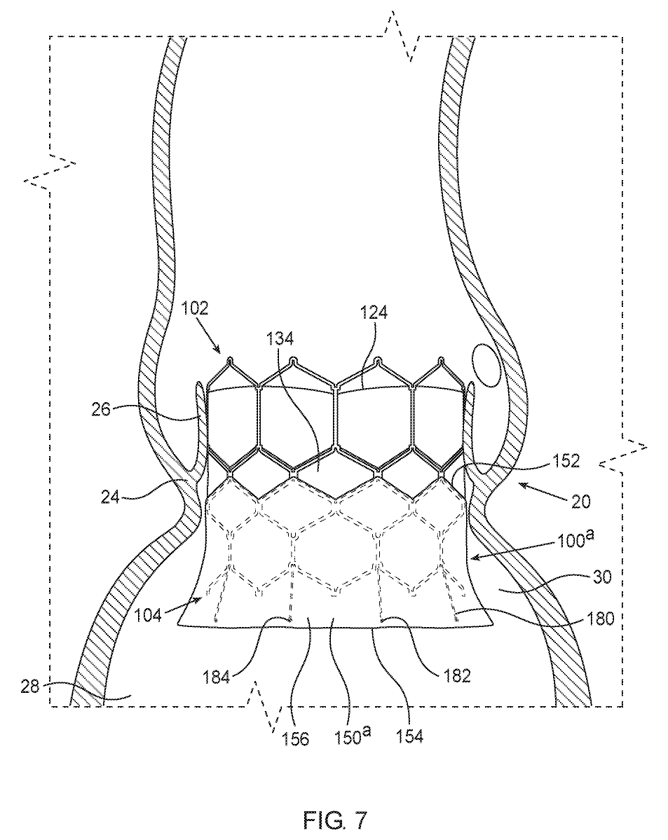

FIG. 7 shows the prosthetic valve of FIGS. 5-6C implanted inside the native heart valve.

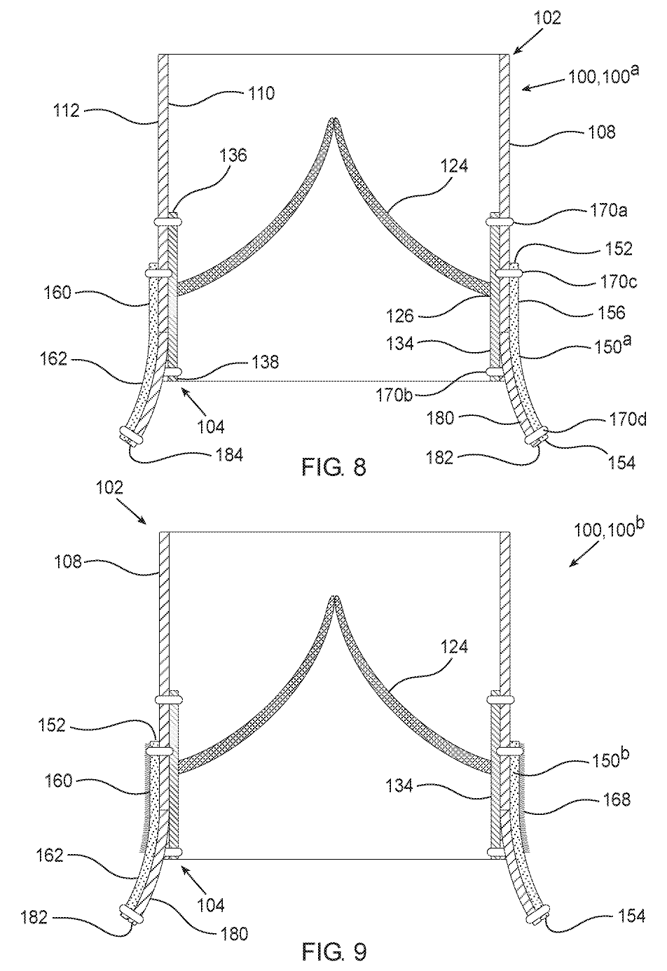

FIG. 8 shows a cross-sectional view of the prosthetic valve of FIG. 7.

FIG. 9 shows a cross-sectional view of an exemplary prosthetic valve including filaments extending from a first portion of an outer skirt thereof.

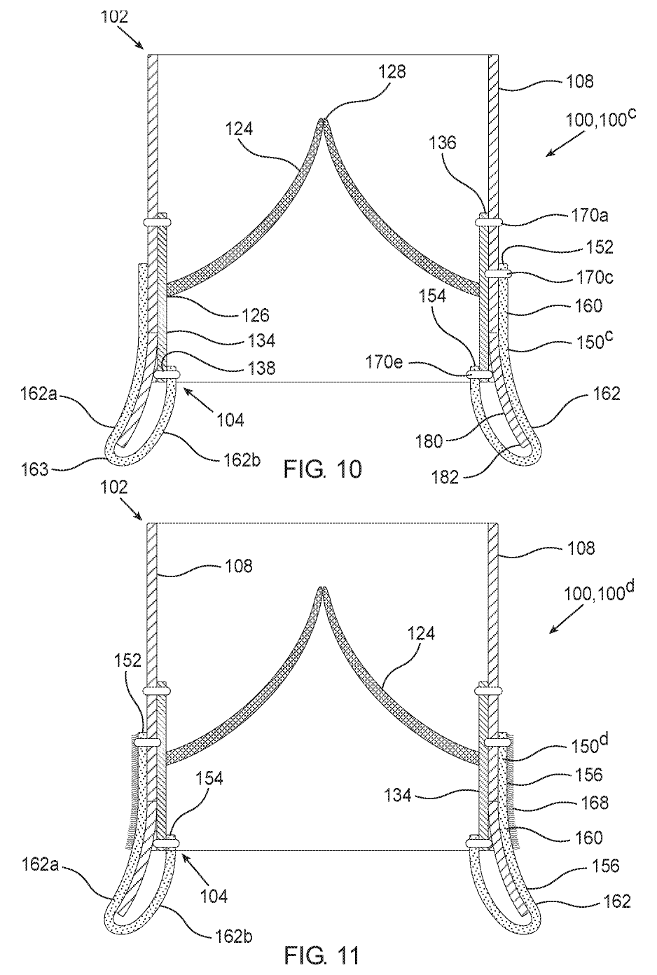

FIG. 10 shows a cross-sectional view of an exemplary prosthetic valve including a second portion of an outer skirt thereof folded over the inflow prongs.

FIG. 11 shows a cross-sectional view of an exemplary prosthetic valve including filaments extending from the first portion of the outer skirt, and a second portion folded over the inflow prongs.

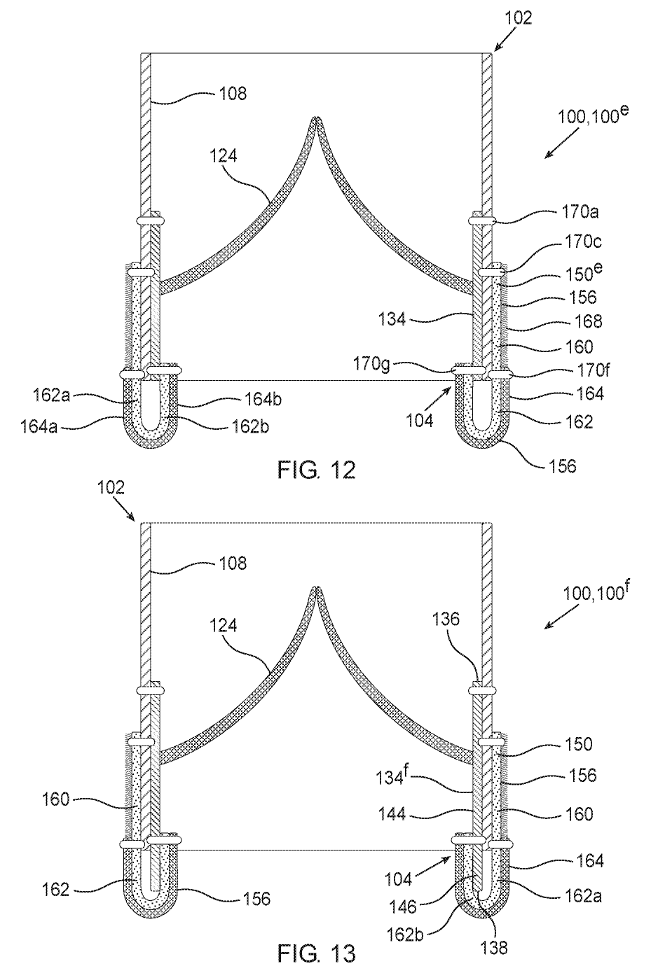

FIG. 12 shows a cross-sectional view of an exemplary prosthetic valve including a reinforcing layers disposed over the second portion of the outer skirt.

FIG. 13 shows a cross-sectional view of an exemplary prosthetic valve including an inner skirt extending distal to the frame.

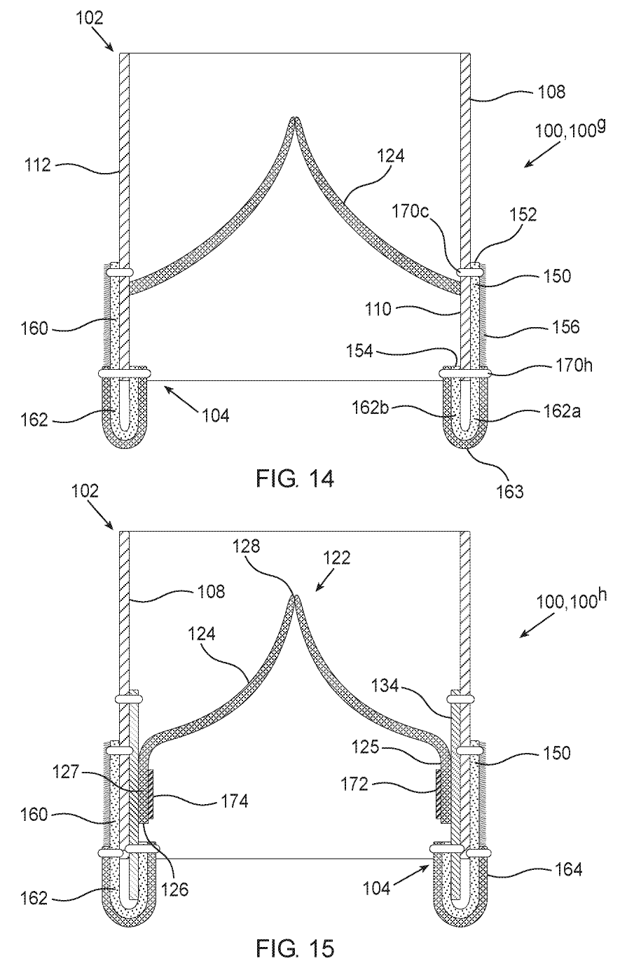

FIG. 14 shows a cross-sectional view of an exemplary prosthetic valve devoid of an inner skirt.

FIG. 15 shows a cross-sectional view of an exemplary prosthetic valve including a leaflet reinforcement strip.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms "have" or “includes” means “comprises”. Further, the terms “coupled”, “connected”, and "attached", as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient’s body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

FIGS. 1A and 1B show perspective views of an exemplary prosthetic valve 10 with and without soft components attached thereto, respectively. The term "prosthetic valve", as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus 202 (see FIG. 2) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (e.g., prosthetic valve 10, 100) may include any prosthetic valve configured to be mounted within the native aortic valve, the native mitral valve, the native pulmonary valve, and the native tricuspid valve.

FIGS. 1A-1B show an example of a prosthetic valve 10, which can be a balloon expandable valve or any other type of valve, illustrated in an expanded state. The prosthetic valve 10 can comprise an annular frame 108 defining an outflow end 102 and an inflow end 104. In some instances, the outflow end 102 is the proximal end of the frame 108, and the inflow end 104 is the distal end of the frame 108. Alternatively, depending for example on the delivery approach of the valve, the outflow end can be the distal end of the frame, and the inflow end can be the proximal end of the frame.

The term "outflow", as used herein, refers to a region of the prosthetic valve through which the blood flows through and out of the prosthetic valve 10.

The term "inflow", as used herein, refers to a region of the prosthetic valve through which the blood flows into the prosthetic valve 10.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The annular frame 108 of prosthetic valve 10 is movable between a radially compressed configuration and a radially expanded configuration, and a valvular structure 122 of the prosthetic valve 10 is mounted within the frame 108. The frame 108 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 108 can be crimped to a radially compressed state on a balloon catheter, and then expanded inside a patient by an inflatable balloon or equivalent expansion mechanism.

In the example illustrated in FIGS. 1A-1B, the frame 108 is an annular, stent-like structure comprising a plurality of intersecting struts 114. In this application, the term "strut" encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 114 may be any elongated member or portion of the frame 108. The frame 108 can include a plurality of strut rungs that can collectively define one or more rows of cells 120. The frame 108 can have a cylindrical or substantially cylindrical shape having a constant diameter from the inflow end 104 to the outflow end 102 as shown, or the frame can vary in diameter along the height of the frame, as disclosed in US Pat. No. 9,155,619, which is incorporated herein by reference.

The end portions of the struts 114 are forming apices 116 at the outflow end 102 and apices 118 at the inflow end 104. The struts 114 can intersect at additional junctions 130 formed between the outflow apices 116 and the inflow apices 118. The junctions 130 can be equally or unequally spaced apart from each other, and/or from the apices 116, 118, between the outflow end 102 and the inflow end 104.

At least some of the struts 114 can be pivotable or bendable relative to each other, so as to permit frame expansion or compression. For example, the frame 108 can be formed from a single piece of material, such as a metal tube, via various processes such as, but not limited to, laser cutting, electroforming, and/or physical vapor deposition, while retaining the ability to collapse/expand radially in the absence of hinges and the like.

A valvular structure 122 can include a plurality of leaflets 124 (e.g., three leaflets), positioned at least partially within the frame 108, and configured to regulate flow of blood through the prosthetic valve 10 from the inflow end 104 to the outflow end 102. While three leaflets 124 arranged to collapse in a tricuspid arrangement, are shown in the example illustrated in FIG. 1A, it will be clear that a prosthetic valve 10 can include any other number of leaflets 124. The leaflets 124 can be formed of pericardial tissue (e.g., bovine pericardial tissue), biocompatible synthetic materials, or various other suitable natural or synthetic materials as known in the art and described in U.S. Patent No. 6,730,118, which is incorporated by reference herein.

Adjacent leaflets 124 can be arranged together to form commissures 132 that are coupled (directly or indirectly) to respective portions of the frame 108, thereby securing at least a portion of the valvular structure 122 to the frame 108. Further details regarding transcatheter prosthetic valves, including the manner in which the valvular structures 122 can be coupled to the frame 108 of the prosthetic valve 10, can be found, for example, in U.S. Patent Nos. 7,393,360, 7,510,575, 7,993,394, 8,652,202, 11,135,056, and 11,096,781, all of which are incorporated herein by reference in their entireties.

As shown for example in FIG. 1A, three separate leaflets 124 can collectively define the valvular structure 122 in some examples. Each leaflet 124 can have a rounded cusp edge 126 (indicated, for example, in FIG. 4) opposite a free edge 128, and a pair of generally oppositely-directed commissure attachment portions separating the cusp edge 126 and the free edge 128. The cusp edge 126 in such cases forms a single scallop. Each leaflet further defines an outer surface 123 facing the frame, and an inner surface 125 facing the central axis Ca.

When such leaflets 124 are coupled to the frame and to each other, the lower edge of the resulting valvular structure 122 desirably has an undulating, curved scalloped shape. By forming the leaflets with this scalloped geometry, stresses on the leaflets 124 are reduced which, in turn, improves durability of the prosthetic valve. Moreover, by virtue of the scalloped shape, folds and ripples at the belly of each leaflet, which can cause early calcification in those areas, can be eliminated or at least minimized. The scalloped geometry also reduces the amount of tissue material used to form the valvular structure, thereby allowing a smaller, more even crimped profile at the inflow end of the valve.

The leaflets 124 can define a non-planar coaptation plane (not annotated) when their free edges 128 co-apt with each other to seal blood flow through the prosthetic valve 10. Leaflets 124 can be secured to one another at their commissure attachment portions to form commissures 132 of the valvular structure 122, which can be secured, directly or indirectly, to structural elements connected to the frame 108 or integrally formed as portions thereof, such as commissure posts or struts, commissure windows, and the like.

In some examples, the prosthetic valve 10 can comprise at least one skirt or sealing member. FIG. 1A shows an example of a prosthetic valve 10 that includes an inner skirt 134, which can be secured to an inner surface 110 of the frame 108. Such an inner skirt 134 can be configured to function, for example, as a sealing member to prevent or decrease perivalvular leakage. An inner skirt 134 can further function as an anchoring region for valvular structure 122 to the frame 108, and/or function to protect the leaflets 124 against damage which may be caused by contact with the frame 108, for example during valve crimping or during working cycles of the prosthetic valve 10. The inner skirt 134 can be coupled to the frame 108 via sutures or another form of coupler.

The prosthetic valve 10 can comprise, in some examples, an outer skirt 150 mounted on the outer surface 112 of frame 108, configure to function, for example, as a sealing member retained between the frame 108 and the surrounding tissue of the native annulus against which the prosthetic valve is mounted, thereby reducing risk of paravalvular leakage (PVL) past the prosthetic valve 10. The outer skirt 150 can be coupled to the frame 108 via sutures or another form of coupler.

Any of the inner skirt 134 and/or outer skirt 150 can be made of various suitable biocompatible materials, such as, but not limited to, various synthetic materials (e.g., PET) or natural tissue (e.g. pericardial tissue). In some cases, the inner skirt 134 can be formed of a single sheet of material that extends continuously around the inner surface 110 of frame 108. In some cases, the outer skirt 150 can be formed of a single sheet of material that extends continuously around the outer surface 112 of frame 108.

The inner skirt 134 extends between a first end 136 and a second end 138 (indicated, for example, in 4), wherein the first end 136 of the inner skirt 134 is closer to the outflow end 102 of the frame 108 than the second end 138. In some examples, the first end 136 can be also referred to as the outflow end 136 of the inner skirt 134, and the second end 138 can be also referred to as the inflow end 138 of the inner skirt 134. The inner skirt 134 can define a first surface 140 facing the central axis Ca of the prosthetic valve, and an opposite second surface 142 (surfaces 140, 142 indicated, for example, in FIG. 4) facing and optionally contacting the inner surface 110 of the frame 108.

The outer skirt 150 extends between a first end 152 and a second end 154, wherein the first end 152 of the outer skirt 150 is closer to the outflow end 102 of the frame 108 than the second end 154. In some examples, the first end 152 can be also referred to as the outflow end 152 of the outer skirt 150, and the second end 154 can be also referred to as the inflow end 154 of the outer skirt 150. The outer skirt 150 can define a first surface 156 facing away from the frame 108, toward the surrounding anatomy when implanted in a patient's body, and an opposite second surface 158 (surfaces 156, 158 indicated, for example, in FIG. 4) facing and optionally contacting the outer surface 112 of the frame 108.

FIG. 2 shows a perspective view of an exemplary delivery assembly 200 that includes a delivery apparatus 202 adapted to deliver a prosthetic device, which can be the prosthetic valve 10 described above, or a prosthetic valve 100 described below with respect to FIGS. 5-14. The delivery apparatus 202 can include a handle 204 and at least one catheter extending therefrom, configured to carry a prosthetic valve 10, 100 in a crimped state through the patient's vasculature. An exemplary delivery assembly 200 comprises an exemplary delivery apparatus 202 configured to carry a balloon expandable prosthetic valve. The delivery apparatus 202 can comprise a balloon catheter 210 having an inflatable balloon 234 mounted on its distal end. A prosthetic device, such as prosthetic valve 10, 100, can be carried in a crimped state over the balloon catheter 210.

In some examples, a delivery apparatus 202 further comprises an outer shaft 208. Optionally, an outer shaft 208 of a delivery apparatus 202 can concentrically extend over the balloon catheter 210.

The outer shaft 208 and the balloon catheter 210 can be configured to be axially movable relative to each other. For example, a proximally oriented movement of the outer shaft 208 relative to the balloon catheter 210, or a distally oriented movement of the balloon catheter 210 relative to the outer shaft 208, can expose the prosthetic valve 10, 100 from the outer delivery shaft 208.

A delivery apparatus 202 can further include a nosecone 224 to facilitate advancement of the delivery apparatus 202 through the patient's vasculature to the site of treatment. A nosecone shaft (concealed from view in FIG. 2) can extend proximally from the nosecone 224 through a lumen of the balloon catheter 210, towards the handle 204.

In FIG. 2, a prosthetic valve 10, 100 is mounted on the balloon 234 and is shown in a crimped state, providing prosthetic valve 10, 100 with a reduced diameter for delivery to the heart via the patient's vasculature. While the prosthetic valve 10, 100 is shown in FIG. 2 as being crimped or mounted on the balloon 234 for delivery to the treatment location, it should be understood that the prosthetic valve can be crimped or mounted at a location different from the location of balloon 234 (e.g., proximal to the balloon 234) and repositioned over the balloon at some time before inflating the balloon and deploying the prosthetic valve. This off-balloon delivery allows the prosthetic valve to be crimped to a lower profile than would be possible if the prosthetic valve was crimped on top of the balloon 234. The lower profile permits the clinician to more easily navigate the delivery apparatus (including the crimped prosthetic valve) through a patient's vasculature to the treatment location. The lower profile of the crimped prosthetic valve can be particularly helpful when navigating through portions of the patient's vasculature which are particularly narrow, such as the iliac artery.

The proximal ends of the balloon catheter 210, the outer shaft 208, and/or the nosecone shaft, can be coupled to the handle 204. During delivery, the handle 204 can be maneuvered by an operator (e.g., a clinician or a surgeon) to axially advance or retract components of the delivery apparatus 202, such as the nosecone shaft, the outer shaft, and/or the balloon catheter 210, through the patient's vasculature and/or along the target site of implantation, as well as to inflate the balloon 234 mounted on the balloon catheter 210, for example to expand a prosthetic valve 10, 100 mounted on the balloon 234, and to deflate the balloon 234 and retract the delivery apparatus 202, for example once the prosthetic valve 10, 100 is mounted in the implantation site.

The handle 204 can include a steering mechanism configured to adjust the curvature of a distal end portion of the delivery apparatus 202. In the illustrated example, the handle 204 includes an adjustment member, such as the illustrated rotatable knob 206a, which in turn is operatively coupled to the proximal end portion of a pull wire (not shown). The pull wire can extend distally from the handle 204 through the outer delivery shaft 208 and has a distal end portion affixed to the outer delivery shaft 208 at or near the distal end of the outer delivery shaft 208. Rotating the knob 206a can increase or decrease the tension in the pull wire, thereby adjusting the curvature of the distal end portion of the delivery apparatus 202. Further details on steering or flex mechanisms for the delivery apparatus can be found in U.S. Pat. No. 9,339,384, which is incorporated by reference herein. The handle 204 can further include an adjustment mechanism including an adjustment member, such as the illustrated rotatable knob 206b. The adjustment mechanism can be configured to adjust the axial position of an ultrasound sensor of the delivery apparatus 202, as will be described in greater detail below. The handle can include additional knobs to control additional components of the delivery apparatus 202, such as positioning members that will be described in greater detail below.

A prosthetic valve 10, 100 can be carried by the delivery apparatus 202 during delivery in a crimped state, and expanded, for example by balloon inflation, to secure it in a native heart valve annulus (such as an aortic annulus) or against a previously implanted prosthetic valve (for example, during valve-in-valve implantation procedures). In some examples, the balloon 234 is secured to a distal end portion of the balloon catheter 210 at its proximal end, while the balloon's distal end can be coupled, directly or indirectly, to another component of the delivery apparatus 202, such as the nosecone 224 or nosecone shaft.

Balloon 234 is configured to transition between a deflated state, shown for example in FIG. 2 and 6A, and an inflated state, shown for example in FIG. 6C. When reaching the site of implantation, the deflated balloon 234, carrying crimped prosthetic valve 10, 100 thereover, can be advanced to the target site to expand the prosthetic valve. Once the prosthetic valve 10, 100 is expanded to its functional diameter within a native annulus or within a previously implanted prosthetic valve, the balloon 234 can be deflated, and the delivery apparatus 202 can be retrieved from the patient's body.

In some examples, the delivery apparatus 202 with the prosthetic valve 10, 100 assembled thereon, can be packaged in a sterile package that can be supplied to end users for storage and eventual use. In some examples, when the leaflets of the prosthetic valve are made from, or include at least an inner core made from, bovine pericardium tissue or other natural or synthetic tissues, the leaflets can be treated during the manufacturing process so that they are completely or substantially dehydrated and can be stored in a partially or fully crimped state without a hydrating fluid. In this manner, the package containing the prosthetic valve and the delivery apparatus, can be free of any liquid. Methods for treating tissue leaflets for dry storage are disclosed in U.S. Pat. Nos. 8,007,992 and 8,357,387, both of which documents are incorporated herein by reference.

FIG. 3A shows a distal portion of an exemplary delivery assembly 200 advanced to a native heat valve for implantation of a prosthetic valve 10 therein. FIG. 3B shows one example of the prosthetic valve 10 implanted in the native heart valve, with the delivery apparatus 202 retrieved from the patient's body. In the implantation procedure illustrated in FIGS. 3A-3B, the prosthetic valve 10 is implanted in a native aortic valve 20 using a transfemoral delivery approach. In other examples, a prosthetic valve 10 can be implanted at other locations (e.g., a mitral valve, a tricuspid valve, and/or a pulmonary valve), within previously-implanted prosthetic valve, and/or using other delivery approaches (e.g., transapical, transaortic, transseptal, etc.).

As shown in FIG. 3A, the delivery assembly 200, including the delivery apparatus 202 and the prosthetic valve 10 retained in a crimped state over the balloon 234, is advanced through the patient's vasculature toward the site of implantation (e.g., the aortic annulus 24) over a guidewire 230. In some examples, as shown in FIG. 3A, the prosthetic valve 10 can be passed through the native leaflets 26 after entering the native aortic valve 20 through the aortic arch, positioning the inflow end 104 past the aortic annulus 24 and in the left ventricle 28, such as in the left ventricle outflow tract (LVOT) 30.

When the prosthetic valve 10 is positioned at a desired position, such as by having the inflow end 104 inside the LVOT 30 at a desired distance below the annulus 24, and/or by having the outflow end 102 inside the aortic root 22 at a desired distance above the annulus 24, the clinician can inflate an expansion balloon 234 to facilitate expansion of the prosthetic valve 10 against the native annulus 24. Once the prosthetic valve 10 is expanded to its functional diameter within a native annulus 24, the balloon 234 can be deflated and the delivery apparatus 202 can be retrieved from the patient's body.

Positioning of the prosthetic valve 10 at a desired position, relative to the native annulus 24, can be accomplished by one or more markers, such as a radiopaque marker (not shown) on the balloon 234, tracked by fluoroscopy of other suitable imaging modalities during the procedure, wherein the delivery apparatus 202 can be maneuvered by the clinician to align the marker with the native annulus 24 or other anatomical structure. During prosthetic valve expansion, the frame foreshortens in a manner that approximates the inflow end 104 and outflow end 102 to each other. The position of the marker can be configured to place the inflow end 104 at a desired distance distally from the plane of the annulus 24, and/or position the outflow end 102 at a desired distance proximally from the plane of the annulus 24, upon valve expansion. FIGS. 3B and 3C show two optional positions of a prosthetic valve 10 implanted inside the native valve 20, wherein, as shown, the prosthetic valve 10 extends deeper into the left ventricle 28, such as deeper into the LVOT 30, in the position illustrated in FIG. 3C, relative to the position illustrated in FIG. 3B. In the lower deployment illustrated in FIG. 3C, the inflow end 104 of the prosthetic valve 10 is distanced farther from the level of the annulus 24, relative to such distance in the higher deployment shown in FIG. 3B.

In conventional prosthetic valves, the inner skirt 134 and the outer skirt 150 are constructed of a porous material, such as woven, braided or knitted fabrics formed from synthetic fibers, such as polyethylene terephthalate (PET) fibers. The porous nature of the skirt material (e.g., having pores greater than 30-50 μm) is designed to encourage cellular ingrowth of the surrounding native tissue while otherwise being substantially impermeable to blood cells in the blood flow through the prosthetic valve. For example, tissue or cells from native leaflets 26 that are in contact with outer skirt 150 can grow into the outer skirt 150 and thereby to the inner skirt 134 on the opposite side of the frame 108. This native tissue ingrowth into skirts 150, 134 can further secure the implanted prosthetic valve 10 within the native anatomy of the patient and reduce risk of PVL.

However, when the inflow end 104 of the prosthetic valve 10 is relatively close to the plane of the native annulus 24 against which the prosthetic valve 10 is deployed, tissue overgrowth may further develop on the inner side of the prosthetic valve 10. FIG. 4 shows a cross-sectional view of an exemplary prosthetic valve 10 after implantation in a native valve 20, schematically illustrating tissue overgrowth 50 developed over time over the inner side of the prosthetic valve 10. Such tissue overgrowth 50 can start at the inflow end 104 of the prosthetic valve 10 and proceed, over time, downstream, towards the region of attachment of the leaflets 124 to the inner skirt 134 or the frame 108 along the scalloped line. This can lead to the formation of pannus on the surfaces of the leaflets 124, which may impede functionality of the valvular structure 122.

As shown in FIG. 4, the second end 154 of the outer skirt 150 of a prosthetic valve 10 can be at or proximate to the inflow end 104 of the frame 108, such that the inflow end 104 of the frame 108 also defines the inflow end of the prosthetic valve 10. In some examples, the second end 138 of the inner skirt 134 can be at or proximate to the inflow end 104 of the frame 108.

The closer the inflow end 104 is to the level of the native annulus 24, the higher is the potential risk of propagation of tissue overgrowth 50 from the inflow end 104 along the inner side of the prosthetic valve, leading to formation of pannus on the surfaces of the leaflets 124. Thus, a lower implantation depth, such as illustrated in FIG. 3C, distancing the inflow end 104 farther from the annulus 24, may reduce the risk of tissue overgrowth and pannus formation on the leaflets 124. However, deeper implantation may be also associated with increased risk of new onset of conduction disturbances, due to potential mechanical injury of the conduction system when the relatively rigid inflow end 104 of the frame 108 is pressed against the membranous septum.

Accordingly, disclosed herein is a prosthetic valve 100, examples of which are described below with respect to FIGS. 5-14, provided with an outer skirt 150 extending below (or distally to) the inflow end 104 of the frame 108. Extending the outer skirt 150 farther in the distal direction, such that the second end 154 of the skirt is distal to the inflow end 104 of the frame, is designed to prevent, or at least reduce, the ingrowth of native tissue propagating from the inflow end 104 along the inner side of the prosthetic valve towards the leaflets 124.

A prosthetic valve 100 according to any of the examples described below with respect to FIGS. 5-14, can be structurally and functionally similar to any example described above with respect to prosthetic valve 10, except that while the second end 154 of the outer skirt 150 of the prosthetic valve 10 is positioned, in the expanded state of the valve, in close proximity to the inflow end 104 of the frame 108, the second end 154 of the outer skirt 150 of a prosthetic valve 100 is positioned, in the expanded state of the valve, distal to the inflow end 104 of the frame 108. Other components and features of prosthetic valve 100, including valvular structure 122 with leaflets 124, frame 108 with struts 114 defining cells 120, and an optional inner skirt 134, can be similar to the same components, with the same component numerals, and features thereof, described above with respect to prosthetic valve 10, and in the interest of brevity will not be described further.

Various exemplary implementations for prosthetic valves 100 can be referred to, throughout the specification, with superscripts, for ease of explanation of features that refer to such exemplary implementations. It is to be understood, however, that any reference to structural or functional features of any apparatus, assembly or component, without a superscript, refer to these features being commonly shared by all specific exemplary implementations that can be also indicated by superscripts. In contrast, features emphasized with respect to an exemplary implementation of device or component, including prosthetic valves 100 and skirts thereof, referred to with a superscript, may be optionally shared by some but not necessarily all other exemplary implementations.

A prosthetic valve 100 can include one or more features configured to support the portion of the outer skirt 150 extending towards the second end 154, in a manner that can prevent, or at least lower, the likelihood of the skirt 150 bending radially inwards (e.g., closer to the central axis Ca) after implantation. At the same time, the portion of the outer skirt 150 extending distal to the inflow end 104 of the frame 108, including any support feature configured to limit inwardly-oriented bending of the skirt, will be less rigid than the frame 108, to mitigate risk of damaging the conductive system when contacting the septum. In some examples, such support features can include a plurality of inflow prongs to which the outer skirt can be coupled. In some examples, such a support feature can include a reinforcing layer disposed over part to the outer skirt.

FIG. 5 shows a portion of an exemplary prosthetic valve 100^{a} with soft components, such as skirts 134, 150 and valvular structure 122, removed from view for clarity. The prosthetic valve 100^{a} can be structurally and functionally similar to any example of prosthetic valve 10 described above, except for including an outer skirt 150 terminating at a second end 154 distal to the inflow end 104 of the frame 108, and a plurality of inflow prongs 180 terminating distally to the inflow end 104, such as at or proximate to the second end 154 of the outer skirt 150.

As shown in FIG. 5, the inflow prongs 180 can extend from the frame 108 and terminate at free ends 182 that are distal to the inflow end 104 of the frame 108. In some examples, the inflow prongs 180 can extend from distal-most non-apical junctions 130a, as shown in the example illustrated in FIG. 5. In some examples, the inflow prongs 180 can extend from the inflow apices 118. In some examples, any inflow prong 180 can extend from any other part of the frame 108, such as any position of an angled strut 114 between a non-apical distal-most junction 130a and a corresponding inflow apex 118.

The outer skirt 150 can extend along the inflow prongs 180, optionally having its second end 154 at or in close proximity to the free ends 182 of the inflow prongs 180. In some examples, the outer skirt 150 can be coupled (e.g., sutured) to the inflow prongs 180. In some examples, the free ends 182 of the inflow prongs 180 comprise eyelets 184 to which the outer skirt 150 can be sutured.

In some examples, the inflow prongs 180 are integrally formed with the frame 108. In some examples, the inflow prongs 180 are provided as separate components that can be affixed (e.g., welded) to the frame 108. The term "integral" or "integrally formed", as used herein, refers to a construction of a component that does not include any welds, fasteners, adhesives or other means for securing separately formed pieces of material to each other.

As shown in FIG. 5, the inflow prongs 180 can extend axially, substantially parallel to the central axis Ca of the prosthetic valve 100, in a free state of the inflow prongs 180. The inflow prongs 180 are made from a material configured to retain the substantially straight configuration, parallel to the central axis Ca and/or to each other, in the absence of external forces acting there-against. This is in contrast to arms that can be alternatively made from shape-memory materials, configured to retain a specific geometry when retained inside an enclosure, such as a capsule of a delivery apparatus, and to assume a free curved or curled configuration when exposed from the enclosure.

In some examples, the inflow prongs 180 are formed of a plastically deformable material, such as stainless steel, cobalt, chromium, titanium, or alloys or combinations of the same (e.g., CoCr alloys), capable of deforming to assume an outwardly-bent shape when an outwardly-directed force is applied thereto.

FIGS. 6A-6C illustrate expansion of a prosthetic valve 100^{a} in greater detail using an inflatable balloon 234. Soft components of the valve 100^{a}, such as skirts 134, 150 or valvular structure 122 are removed from view in FIGS. 6A-6C for clarity. FIG. 6A shows the prosthetic valve 100^{a} crimped onto the balloon 234, with the inflow prongs 180 axially extending, in their free state, substantially parallel to the central axis Ca and to each other. Because the struts 114 of the frame 108 are interconnected at various angles, so as to define rows of closed cells 120, the frame 108 is relatively stiffer or more resistant to radial expansion, compared to any inflow prong 180 which is connected to the frame 108 at a single junction, and is unrestricted at the opposite free end 182. When the balloon 234 is partially inflated, it can form a "dog bone" shape in which the proximal and distal end portions of the balloon 234 which are not constrained by the frame 108, inflate to a greater degree than the portion of the balloon around which the frame 108 is crimped. During this phase, the free-ended inflow prongs 180 can bend outwardly to a greater diameter than the remainder of the frame 108, as shown in FIG. 6B, due to the relatively greater resistance of the frame 108 to expansion.

In examples in which the inflow prongs 180 are plastically deformable, they retain their outwardly bent configuration, relative to the rest of the frame, such that even when the frame 108 is fully expanded on the balloon 234, the support arms retain their angled configuration relative to the inflow end 104 of the frame 108, collectively defining an outwardly flared configuration, as shown in FIG. 6C. The inflow prongs 180 terminate at free ends 182 that collectively define a greater diameter than the diameter defined by the inflow apices 118 in such examples.

FIG. 7 shows the prosthetic valve 100^{a} implanted inside the native heart valve 20, wherein the second end 154 of the outer skirt 150^{a}, positioned at the level of the free ends 182 of the inflow prongs 180, is distal to the inflow end 104 of the frame 108, and/or from the inflow apices 118. Since the lower second end 154 of the skirt 150^{a} defines an inflow end of the prosthetic valve 100^{a} that is lower than the inflow end 104 of the frame 108, the prosthetic valve 100^{a} can be deployed at higher position, while the inflow end of the valve 100^{a}, defined by the second end 154 of the elongated skirt 150^{a}, extends farther from the annulus 24, thereby mitigating the risk of tissue overgrowth and pannus formation on the leaflets, while at the same time avoiding from inflicting damage to the conduction system, since the inflow prongs 180 are significantly less rigid than the frame 108, and thus will not apply the same pressure that can damage the septal tissue and conduction paths passing therealong, when more rigid portion of the frame 108 are pressed thereagainst.

In the example illustrated in FIG. 7, the outer skirt 150^{a} is shown to assume a flared configuration, due to the optional bending of the inflow prongs 180 radially outward during balloon inflation, as described above with respect to FIGS. 6A-6C. It is to be understood, however, that in some examples, the inflow prongs 180 may be rigid enough to retain a relatively straight axial configuration even during balloon inflation, as long as their rigidity is still smaller than that of the frame 108 (such as along the inflow end 104 of the frame 108), to reduce the risk of applying a damaging mechanical force on the conduction pathways of the septum when pressed thereagainst. Moreover, even when the inflow prongs 180 are configured to bend outwards during balloon inflation, it is to be understood that such bending is relatively modest, and that the inflow prongs 180 are not configured to curl or assume U-shaped configurations. Stated otherwise, the free ends 182 of the inflow prongs 180 will remain the distal-most parts or ends of the inflow prongs 180 at all times, prior to, as well as after, full deployment and prosthetic valve expansion.

FIG. 8 shows a cross-sectional view of an exemplary prosthetic valve 100^{a} of the type shown in FIG. 7. The outer skirt 150^{a} can include a first portion 160 extending distally from the first end 152 of the outer skirt 150^{a}, and a second portion 162 extending between the first portion 160 and the second end 154 of the skirt 150^{a}. In some examples, the first portion 160 can be disposed around at least a portion of the frame 108. In some examples, the second portion 162 can be disposed around, and optionally attached to, the inflow prongs 180. In some examples, the second portion 162 can continuously extend from the first portion 160. In some examples, as illustrated in FIG. 8, the outer skirt 150^{a} can have a uniform first surface 156 collectively defined over the first 160 and second 162 portions.

In some examples, an inner skirt 134 can be coupled by sutures 170a connecting the first end 136 to the frame 108, and by sutures 170b connecting the second end 138 to the frame 108, optionally at or proximate to the inflow end 104. In some examples, the outer skirt 150 can be coupled by sutures 170c connecting the first end 152 to the frame 108, and by sutures 170d connecting the second end 154 to the inflow prongs 180, optionally at or proximate to the free ends 182. Additional sutures may be used to couple any of the skirts to the frame and/or the inflow prongs. In some examples, the sutures 170c extend through the outer skirt 150 and the frame 108, without further extending through the inner skirt 134. In some examples, the sutures 170c extend through the outer skirt 150, the frame 108, and the inner skirt 134. In some examples, the sutures 170b extend through the inner skirt 134 and the frame 108, without further extending through the outer skirt 150. In some examples, the sutures 170c extend through the inner skirt 134, the frame 108, and the outer skirt 150.

FIG. 9 is a cross-sectional view of an exemplary prosthetic valve 100^{b}. Prosthetic valve 100^{b} is similar to any example described herein for prosthetic valve 100^{a}, except that a portion of the first surface 156 of the outer skirt 150^{b}of prosthetic valve 100^{b} is configured to encourage tissue overgrowth. In some examples, the first surface 156 extending over the first portion 160 of the outer skirt 150^{b}is configured to encourage tissue overgrowth to a greater extent than the first surface 156 extending over the second portion 162.

Relative thrombogenicity or tissue adherence values between articles that can include different materials, or include the same material but having different surface textures, can be determined, for example, according to the regulation (EU) 2017/745 of the European parliament and of the council on medical devices. The regulation refers to ISO10993–4–2017, and includes measurements to be performed in order to determine three main parameters relating to thrombogenicity: (1) Thrombin generation, as measured by ELISA (Enzyme-Linked Immunosorbent Assay) for Thrombin–antithrombin complex and Prothrombin fragment F1+2; (2) Fibrin generation as measured by ELISA for Fibrinopeptide A; and (3) Intrinsic pathway (FXII) as measured by PTT (Partial Thromboplastin Time) test.

In some examples, the first surface 156 extending over the first portion 160 of the outer skirt 150^{b} is a textured surface. In some examples, the first surface 156 extending over the first portion 160 of the outer skirt 150^{b} comprises outwardly-oriented filaments 168 configured to encourage tissue ingrowth. In some examples, the first surface 156 extending over the second portion 162 comprises a thromboresistant surface. The term “thromboresistant” refers to a material or an article (e.g., an implant or a part thereof), which is substantially resistant to biological damaging caused by platelet adhesion, thrombus formation and/or tissue ingrowth in vitro and/or in vivo. Specifically, “thromboresistant surface” refers to a surface texture which imparts thromboresistance to an article, such as a skirt or a portion thereof. Some materials may be formed to have relatively smooth surfaces that resist tissue adherence or growth thereon, including resistance to adherence of thrombus or blood clots to the surface, and/or resistance to neointimal tissue overgrowth. Exemplary materials that can have thromboresistant surfaces include, but are not limited to, PTFE (polytetrafluoroethylene) and TPU (thermoplastic polyurethane).

In some examples, the first portion 160 of the outer skirt 150^{b} comprises a plurality of filaments 168 extending outwardly therefrom, while the second portion 162 is devoid of outwardly-extending filaments 168. In some examples, the first portion 160 and the second portion 162 can be made of the same material, together defining a unitary outer skirt portion, wherein the second portion 162 can be further coated by a thromboresistant coating, for example along its first surface 156. In some examples, the first portion 160 and the second portion 162 can be made of the same material, together defining a unitary outer skirt portion, wherein the first portion 160 comprises filaments 168 or is otherwise textured along the first surface to encourage tissue ingrowth, while the second portion 162 is devoid of filaments or any other tissue-growth encouraging texture, such that the first surface 156 along second portion 162 is not necessarily thromboresistant, but may be merely designed not to actively encourage ingrowth. In some examples, the second surface 158 of the outer skirt 150 is a thromboresistant surface along at least part of the outer skirt, such as along the second portion 162.

FIG. 10 is a cross-sectional view of an exemplary prosthetic valve 100^{c}. Prosthetic valve 100^{c} is similar to any example described herein for prosthetic valve 100^{a}, except that the second portion 162 does not necessarily terminate at the free ends 182 of inflow prongs 180, but is rather folded around the free ends 182 and proximally extends along the inner side of the inflow prongs 180 back towards the frame 108. In some examples, the second portion 162 of the outer skirt 150^{c} can be coupled by sutures 170e connecting the second end 154 to the inner skirt 134, such as long the second end 138 of the inner skirt, and/or connecting the second end 154 to the frame 108, such as along its inflow end 104. When folded over itself, the second portion 162 can have an outer section 162a that distally extends from the first portion 160 to a distal fold 163, and an inner section 162b that proximally extends from the distal fold 163 towards the inflow end 104 of the frame 108, such that the first surface 156 of the skirt 150 along the first section 162a is oriented radially outwards, and the first surface 156 along the second section 162b is oriented radially inwards (e.g., towards central axis Ca). In the example illustrated in FIG. 10, the inflow end of the prosthetic valve 100^{c} is defined by the distal fold 163 of the outer skirt 150^{c}around the inflow prongs 180.

FIG. 11 is a cross-sectional view of an exemplary prosthetic valve 100^{d}. Prosthetic valve 100^{d} is similar to any example described herein for prosthetic valve 100^{c}, having the second portion 162 of the outer skirt 150^{d}folded over the inflow prongs 180, except that a portion of the first surface 156 of the outer skirt 150^{d} of prosthetic valve 100^{b} is configured to encourage tissue overgrowth. The surfaces of the outer skirt 150^{d} can be implemented in a similar manner to any of the examples described above with respect to FIG. 9. For example, the first portion 160 of the outer skirt 150^{d} can include a textured surface, optionally including filaments 168 as described above with respect to the outer skirt 150^{b}, while the surface of the second portion 162 can be devoid of filaments or other tissue-growth encouraging textures.

FIG. 12 is a cross-sectional view of an exemplary prosthetic valve 100^{e}. Prosthetic valve 100^{e} can be structurally and functionally similar to any example of prosthetic valve 10 described above, except for including an outer skirt 150 that extends distally past the inflow end 104 of the frame 108, wherein at least part of outer skirt portions extending past the frame's inflow end 104, such as a second portion 162 of the outer skirt 150, further includes a reinforcing layer 164 configured to limit inwardly-oriented bending of the second portion 162 of the outer skirt.

The outer skirt 150^{e} is shown to include a first portion 160 extending between the first end 152 of the skirt 150^{e} and the inflow end 104 of the frame 108, and a second portion 162 distal to the inflow end 104 of the frame 108. The second portion 162 can extend distally from the first portion 160, such as from the inflow end 104 of the frame 108, fold over itself, and extend proximally from the fold of the skirt towards the frame 108, such as back towards the inflow end 104 of the frame 108, optionally into the inner side of the frame 108. In some examples, the second portion 162 of the outer skirt 150^{e} can be coupled by sutures 170g connecting the second end 154 to the inner skirt 134, such as long the second end 138 of the inner skirt, and/or to the frame 108, such as along its inflow end 104. In the example illustrated in FIG. 12, the inflow end of the prosthetic valve 100^{e} is defined by the distal fold of the outer skirt 150^{e}.

A reinforcing layer 164 can be added over the second portion 162, which can be in the form of a coating, or a separately-formed layer that can be coupled to the second portion 162 of the outer skirt 150^{e}, such as by stitching, fusing, adhering, and the like. The reinforcing layer impart greater rigidity o bending resistance to the second portion 162 of the outer skirt 150^{e}. The reinforcing layer can be made of a different material than that of the remainder of the outer skirt 150^{e}, or may be formed from a similar material but resulting in an overall increased thickness of the second portion 162. While the reinforcing layer 164 is shown in the example illustrated in FIG. 12 to be disposed over the first surface 156 of the outer skirt 150^{e}, it is to be understood that a reinforcing layer can be similarly disposed over the second surface 158, or both first 156 and second 158 surfaces of the second portion 162 of the outer skirt 150^{e}.

The reinforcing layer 164 serves as a support feature configured to limit inwardly-oriented bending of the second portion 162 of the outer skirt 150 after implantation, to prevent it from restricting blood inflow into the valve, or from forming a "step"-like disturbance that may result in flow abnormalities. When the second portion 162 is bent over itself, its overall thickness may be doubled, thereby increasing the overall bending resistance. Utilization of a reinforcing layer 164 as a skirt-support feature can obviate the need for inflow prongs 180 of the type described above, such that a prosthetic valve 100^{e}, as shown in FIG. 12 (as well as prosthetic valves with skirt reinforcing layers 164 shown in FIGS. 13-14) can be devoid of inflow prongs 180. Nevertheless, it is to be understood that any exemplary prosthetic valve 100 described herein to include a reinforcing layer 164 disposed over the second portion 162, can be used in combination with inflow prongs 180 according to any example described above, such that both the inflow prongs 180 and the reinforcing layer 164 will limit inwardly oriented bending of the second portion 162 of the outer skirt 150.

While the second portion 162 is illustrated and described to fold over itself, it is to be understood that in some examples, a second portion 162 covered by a reinforcing layer 164 can extend distal to the inflow end 104 of the frame 108 without necessarily being overbent, such as by terminating with a free-ended second end 154 of the outer skirt 150, positioned distal to the inflow end 104 of the frame 108, the second end 154 defining in such cases the inflow end of the prosthetic valve 100. This can be implemented, for example, when the reinforcing layer 164 imparts sufficient rigidity to limit inward-bending of a second portion 162 unattached, at the second end 154, to any other component of the frame 108 or inner skirt 134.

While a reinforcing layer is disposed over the second portion 162, the first portion 160 of the outer skirt 150^{e} can remain, in some examples, devoid of the reinforcing layer. The first portion 160 of the outer skirt 150^{e} is disposed around a portion of the prosthetic valve 100^{e} that includes also the frame 108, the lower part of the leaflet 124, and optionally the inner skirt 134. Adding another layer, such as reinforcing layer 164, to the first portion 160 of the outer skirt 150, can increased the thickness of the first portion 160 in this portion of the prosthetic valve 100, which in turn will increase the overall crimped profile of the valve 100 in this region. The diameter of the crimped profile of a prosthetic valve is an important design parameter of transcatheter prosthetic valves, because it directly influences the physician's ability to advance the prosthetic heart valve through the femoral artery or vein. More particularly, a smaller profile allows for treatment of a wider population of patients, with enhanced safety.

Since reinforcement is not required along the first portion 160 of the outer skirt 150^{e}, adding such a layer in a uniform manner along the entire outer skirt, including the first portion 160, will serve no purpose while unnecessarily increasing the crimped profile along this critical region of the valve. Thus, including of a reinforcing layer, limited to the second portion 162 of the outer skirt 150, will focus on the region in which this layer may be of advantage, while avoiding any increase in outer diameter at critical regions of the valve. Because the second portion 162 of the outer skirt 150 extends distal to the frame 108 and the leaflets 124, the second portion 162 can be safely thickened without increasing the maximal outer diameter of the valve 100 when crimped over the balloon 234.

In some examples, the second portion 162 can extend between sutures 170f, coupling a part of the outer skirt 150^{e} disposed radially outward to the frame 108, to the inflow end 104 of the frame 108, and sutured 170g, coupling part of the outer skirt 150^{e} disposed radially inwards to the frame 108, also to the inflow end 104 of the frame 108, sandwiching the frame 108, and optionally inner skirt 134, between outer and inner part of the outer skirt 150^{e}. While separate sutures 170f and 170g are illustrated in FIG. 12, in some examples, a single line of sutures 170 can extend through both inner and outer parts of the outer skirt 150^{e}, as well as through the frame 108 and optionally through inner skirt 134.

In some examples, the first portion 160 is configured to encourage tissue overgrowth, such as by including filaments 168 or having its first surface 156 otherwise textured, while the second portion 162, including reinforcing layer 164, is not configured to encourage tissue growth or adherence, and may include, in some examples, a thromboresistant surface. A tissue overgrowth encouraging first portion 160 and thromboresistant or otherwise tissue growth non-encouraging second portion 162 can be implemented in the same manner described above with respect to FIG. 9, mutatis mutandis.

FIG. 13 is a cross-sectional view of an exemplary prosthetic valve 100^{f}. Prosthetic valve 100^{f} is similar to any example described herein for prosthetic valve 100^{e}, except that the inner skirt 134^{f}extends distally past the inflow end 104 of the frame 108. The inner skirt 134^{f}can include a first portion 144, extending between the first end 136 of the skirt 134^{f} and the inflow end 104 of the frame, and a second portion 146 extending between the inflow end 104 of the frame 108 and the second end 138 of the skirt 134^{f}. When the outer skirt 150 is folded over itself, it can form a pocket distal to the inflow end 104, into which the second portion 146 of the inner skirt 134^{f}can extend, in the expanded state of the prosthetic valve 100. Such a configuration can add the thickness of the inner skirt 134^{f}to the reinforced portion of the outer skirt 150 below the frame 108, thereby further increasing the bending resistance of the skirts distal to the frame 108.

While an inner skirt 134^{f} is shown to extend into a pocket formed by the second portion 162 of the outer skirt 150 in FIG. 13, it is to be understood that the inner skirt 134^{f} can extend distally to the inflow end 104 of the frame 108 also when the outer skirt 150 is not folded over itself. Moreover, while an inner skirt 134^{f} extending distally to the frame 108 is illustrated in FIG. 13 in combination with an outer skirt 150 reinforced by a reinforcing layer 164, it is to be understood that an elongated inner skirt 134 that includes a second portion 146 extending past the inflow end 104 of the frame 108, can be similarly used with any other exemplary valve 100 disclosed above with respect to FIGS. 8-12. For example, when used in combination with a prosthetic valve that includes inflow prongs 180, such as any of the exemplary prosthetic valve 100^{a}, 100^{b}, 100^{c}, 100^{d}, the second portion 146 of the inner skirt 134 can extend along an inner side of the inflow prongs 180, and can be optionally sutured to the inflow prongs 180.

FIG. 14 is a cross-sectional view of an exemplary prosthetic valve 100^{g}. Prosthetic valve 100^{f} is similar to any example described herein for prosthetic valve 100^{g}, except that prosthetic valve 100^{g} is devoid of an inner skirt 134. In some examples, leaflets 124 of the prosthetic valve 100^{g} can be directly sutured to struts 114 of the frame 108, and/or to the first portion 160 of the outer skirt 150. Sutures 170h can extend coupled the parts of the outer skirt 150 on both sides of the frame 108 to the inflow end 104 of the frame 108. through both inner and outer parts of the outer skirt 150^{e}, as well as through the frame 108 and optionally through inner skirt 134. While a prosthetic valve 100 devoid of an inner skirt is illustrated in FIG. 14 to include an outer skirt 150 reinforced by a reinforcing layer 164, it is to be understood that an inner skirt can be similarly removed from any other exemplary valve 100 disclosed above with respect to FIGS. 8-12, such as any of exemplary prosthetic valves 100^{a}, 100^{b}, 100^{c}, 100^{d}.

FIG. 15 is a cross-sectional view of an exemplary prosthetic valve 100^{h}. Prosthetic valve 100^{h} is similar to any example described herein for prosthetic valve 100, except that prosthetic valve 100^{h} further comprises a reinforcement strip disposed over an inner surface 125 of the leaflet 124, along a leaflet inflow portion 127 that extends proximally from the cusp edge 128. Sutures (not shown) can be passed through the reinforcement strip 172 and the leaflet inflow portion 128 to couple the leaflet 124 to the inner skirt 134 and/or to the frame 108. In some examples, the reinforcement strip 172 can be made from PET or other type of fabric. In some examples, the reinforcement strip 172 can be made of a thromboresistant material, such as TPU or PTFE. In some examples, the reinforcement strip 172 has a inner surface 174, facing the central axis Ca, which is can be a thromboresistant surface. For example, the reinforcement strip 172 can be coated or otherwise include a thromboresistant layer attached thereto, that defines an inner surface 174 which is thromboresistant.

While the reinforcement strip 172 is illustrated in FIG. 15 to be separated from the second portion 162 of the outer skirt 150 and/or a reinforcing layer 164 attached thereto, it is to be understood that this is shown by way of illustration and not limitation. In some examples, the reinforcement strip 172 can extend distally from the cusp edge 126 of the leaflet 124 and cover, as well as being optionally attached to, at least part of the second portion 162 of the outer skirt 150 and/or at least part of reinforcing layer 164. In some examples, the second portion 162 of the outer skirt 150 and/or reinforcing layer 164 can extend proximally from the inflow end 104 of the frame 108 so as to cover, as well as optionally be attached to, at least part of the reinforcement strip 172.

While a prosthetic valve 100 that includes a reinforcement strip 172 is illustrated in FIG. 15 to include an outer skirt 150 reinforced by a reinforcing layer 164, and an inner skirt 134 that is shown to have a second portion thereof extending past the inflow end 102 of the frame 108, it is to be understood that a reinforcement strip 172 can be added to any other exemplary valve 100 disclosed above with respect to FIGS. 8-14, such as any of exemplary prosthetic valves 100^{a}, 100^{b}, 100^{c}, 100^{d}, 100^{e}, 100^{g}.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve comprising:

a plastically-expandable frame extending between an inflow end and an outflow end, and movable between a radially compressed state and a radially expanded state;

a plurality of plastically deformable inflow prongs attached to the frame and terminating at free ends positioned distal to the inflow end of the frame in the expanded state; and

an outer skirt comprising a first portion disposed around the frame, and a second portion extending distal to the frame and disposed around the inflow prongs;

wherein the inflow prongs are configured to limit a radially-inward oriented bending of the second portion of the outer skirt supported by the inflow prongs.

Example 2. The prosthetic valve of any example herein, particularly of example 1, wherein the prosthetic valve is a balloon expandable valve.

Example 3. The prosthetic valve of of any example herein, particularly of example 1 or 2, wherein the inflow prongs are configured to maintain an unbent configuration in a free state thereof.

Example 4. The prosthetic valve of any example herein, particularly of any one of examples 1 to 3, wherein the inflow prongs are configured to bend radially outwards upon inflation of a balloon over which the prosthetic valve is crimped.

Example 5. The prosthetic valve of any example herein, particularly of example 4, wherein the second portion of the outer skirt is configured to assume an outwardly flared configuration in the expanded state.

Example 6. The prosthetic valve of any example herein, particularly of any one of examples 1 to 5, wherein the free ends of the inflow prongs define a distal-most end of the inflow prongs in the expanded state.

Example 7. The prosthetic valve of any example herein, particularly of any one of examples 1 to 6, wherein the second portion of the outer skirt is coupled to the inflow prongs.

Example 8. The prosthetic valve of any example herein, particularly of example 7, wherein the second portion of the outer skirt is sutured to eyelets at the free ends of the inflow prongs.

Example 9. The prosthetic valve of any example herein, particularly of any one of examples 1 to 8, wherein the inflow prongs are devoid of shape-memory materials.

Example 10. The prosthetic valve of any example herein, particularly of any one of examples 1 to 9, wherein the first portion of the outer skirt comprises a textured surface configured to encourage tissue growth thereover.

Example 11. The prosthetic valve of any example herein, particularly of any one of examples 1 to 10, wherein the first portion of the outer skirt comprises a plurality of outwardly extending filaments.

Example 12. The prosthetic valve of any example herein, particularly of any one of examples 1 to 11, wherein the second portion of the outer skirt is devoid of textured surfaces that may encourage tissue growth thereover.

Example 13. The prosthetic valve of any example herein, particularly of any one of examples 1 to 12, wherein the second portion of the outer skirt is devoid of outwardly extending filaments.

Example 14. The prosthetic valve of any example herein, particularly of any one of examples 1 to 13, wherein the second portion of the outer skirt comprises a thromboresistant surface.

Example 15. The prosthetic valve of any example herein, particularly of any one of examples 1 to 14, wherein the outer skirt extends between a first end and a second end, wherein the first end of the outer skirt is coupled to the frame between the inflow end and the outflow end of the frame.

Example 16. The prosthetic valve of any example herein, particularly of example 15, wherein the second end of the outer skirt is coupled to the inflow prongs.

Example 17. The prosthetic valve of any example herein, particularly of example 15 or 16, wherein the second end of the outer skirt is distal to the inflow end of the frame.

Example 18. The prosthetic valve of any example herein, particularly of any one of examples 15 to 17, wherein the second end of the outer skirt defines an inflow end of the prosthetic valve, which is distal to the inflow end of the frame in the expanded state.

Example 19. The prosthetic valve of any example herein, particularly of example 15, wherein the second end of the outer skirt is coupled to the inflow end of the frame.

Example 20. The prosthetic valve of any example herein, particularly of example 1 or 19, wherein the second portion of the outer skirt is folded around the free ends of the inflow prongs.

Example 21. The prosthetic valve of any example herein, particularly of example 20, wherein a fold of the outer skirt around the inflow prongs defines an inflow end of the prosthetic valve, which is distal to the inflow end of the frame in the expanded state.

Example 22. The prosthetic valve of any example herein, particularly of any one of examples 1 to 21, wherein the inflow prongs are less rigid than the frame.

Example 23. The prosthetic valve of any example herein, particularly of any one of examples 1 to 22, wherein the inflow prongs are less resistant to radial bending than the frame.

Example 24. The prosthetic valve of any example herein, particularly of any one of examples 1 to 23, wherein the inflow prongs and the frame are integrally formed.

Example 25. The prosthetic valve of any example herein, particularly of any one of examples 1 to 24, further comprising an inner skirt disposed around an inner surface of the frame.

Example 26. The prosthetic valve of any example herein, particularly of any one of examples 1 to 25, further comprising a valvular structure mounted inside the frame and comprising a plurality of leaflets.

Example 27. The prosthetic valve of any example herein, particularly of example 26, wherein the plurality of leaflets comprises three leaflets.

Example 28. The prosthetic valve of any example herein, particularly of example 26 or 27, further comprising a reinforcement strip disposed over an inner surface of the plurality of leaflets.

Example 29. The prosthetic valve of any example herein, particularly of example 28, wherein the reinforcement strip is made of a thromboresistant material.

Example 30. The prosthetic valve of any example herein, particularly of example 28, wherein the reinforcement strip comprises a thromboresistant inner surface.

Example 31. A prosthetic valve comprising:

a frame extending between an inflow end and an outflow end, and movable between a radially compressed state and a radially expanded state;

an outer skirt comprising a first portion disposed around the frame and a second portion extending distal to the inflow end of the frame; and

a reinforcing layer disposed over the second portion of the outer skirt;

wherein the reinforcing layer is configured to limit a radially-inward oriented bending of the second portion of the outer skirt; and

wherein the reinforcing layer is not disposed over the first portion of the outer skirt.

Example 32. The prosthetic valve of any example herein, particularly of example 31, wherein the reinforcing layer comprises a coating.

Example 33. The prosthetic valve of any example herein, particularly of example 31, wherein the reinforcing layer comprises a separately-formed layer coupled to the second portion of the outer skirt.

Example 34. The prosthetic valve of any example herein, particularly of any one of examples 31 to 33, wherein the reinforcing layer and the outer skirt are made of different materials.

Example 35. The prosthetic valve of any example herein, particularly of any one of examples 31 to 34, wherein a thickness of the second portion of the outer skirt, combined with the reinforcing layer, is greater than a thickness of the first portion of the outer skirt.

Example 36. The prosthetic valve of any example herein, particularly of any one of examples 31 to 35, wherein an outer diameter of the prosthetic valve defined at a level of the second portion of the outer skirt is not greater than an outer diameter of the prosthetic valve defined at a level of the first portion of the outer skirt, in the radially compressed state.

Example 37. The prosthetic valve of any example herein, particularly of any one of examples 31 to 36, wherein the reinforcing layer is stiffer than the outer skirt.

Example 38. The prosthetic valve of any example herein, particularly of any one of examples 31 to 37, wherein the first portion of the outer skirt comprises a textured surface configured to encourage tissue growth thereover.

Example 39. The prosthetic valve of any example herein, particularly of any one of examples 31 to 38, wherein the first portion of the outer skirt comprises a plurality of outwardly extending filaments.

Example 40. The prosthetic valve of any example herein, particularly of any one of examples 31 to 39, wherein the second portion of the outer skirt is devoid of textured surfaces that may encourage tissue growth thereover.

Example 41. The prosthetic valve of any example herein, particularly of any one of examples 31 to 40, wherein the second portion of the outer skirt is devoid of outwardly extending filaments.

Example 42. The prosthetic valve of any example herein, particularly of any one of examples 31 to 41, wherein the reinforcing layer is devoid of textured surfaces that may encourage tissue growth thereover.

Example 43. The prosthetic valve of any example herein, particularly of any one of examples 31 to 42, wherein the reinforcing layer is devoid of outwardly extending filaments.

Example 44. The prosthetic valve of any example herein, particularly of any one of examples 31 to 43, wherein the second portion of the outer skirt comprises a thromboresistant surface.

Example 45. The prosthetic valve of any example herein, particularly of any one of examples 31 to 44, wherein the reinforcing layer comprises a thromboresistant surface.

Example 46. The prosthetic valve of any example herein, particularly of any one of examples 31 to 45, wherein the outer skirt extends between a first end and a second end, wherein the first end of the outer skirt is coupled to the frame between the inflow end and the outflow end of the frame.

Example 47. The prosthetic valve of any example herein, particularly of example 46, wherein the second end of the outer skirt is distal to the inflow end of the frame.

Example 48. The prosthetic valve of any example herein, particularly of example 46 or 47, wherein the second end of the outer skirt defines an inflow end of the prosthetic valve, which is distal to the inflow end of the frame in the expanded state.

Example 49. The prosthetic valve of any example herein, particularly of example 46, wherein the second end of the outer skirt is coupled to the inflow end of the frame.

Example 50. The prosthetic valve of any example herein, particularly of any one of examples 31-46 or 49, wherein the second portion of the outer skirt is folded over itself.

Example 51. The prosthetic valve of any example herein, particularly of example 50, wherein a fold of the second portion of the outer skirt defines an inflow end of the prosthetic valve, which is distal to the inflow end of the frame in the expanded state.

Example 52. The prosthetic valve of any example herein, particularly of any one of examples 31 to 48, further comprising an inner skirt disposed around an inner surface of the frame.

Example 53. The prosthetic valve of any example herein, particularly of example 50 or 51, further comprising an inner skirt that comprises a first portion disposed around an inner surface of the frame, and a second portion extending distally from the inflow end of the frame.

Example 54. The prosthetic of any example herein, particularly of example 53, wherein the second portion of the inner skirt extends into a pocket formed by the second portion of the outer skirt.

Example 55. The prosthetic valve of any example herein, particularly of any one of examples 31 to 54, further comprising a valvular structure mounted inside the frame and comprising a plurality of leaflets.

Example 56. The prosthetic valve of any example herein, particularly of example 55, wherein the plurality of leaflets comprises three leaflets.

Example 57. The prosthetic valve of any example herein, particularly of example 55 or 56, further comprising a reinforcement strip disposed over an inner surface of the plurality of leaflets.

Example 58. The prosthetic valve of any example herein, particularly of example 57, wherein the reinforcement strip is made of a thromboresistant material.

Example 59. The prosthetic valve of any example herein, particularly of example 57, wherein the reinforcement strip comprises a thromboresistant inner surface.

Example 60. The prosthetic valve of any example herein, particularly of any one of examples 31 to 59, wherein the frame is plastically-expandable.

Example 61. The prosthetic valve of any example herein, particularly of any one of examples 31 to 60, wherein the prosthetic valve is a balloon expandable valve.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve comprising:
a plastically-expandable frame extending between an inflow end and an outflow end, and movable between a radially compressed state and a radially expanded state;
a plurality of plastically deformable inflow prongs attached to the frame and terminating at free ends positioned distal to the inflow end of the frame in the expanded state; and
an outer skirt comprising a first portion disposed around the frame, and a second portion extending distal to the frame and disposed around the inflow prongs;
wherein the inflow prongs are configured to limit a radially-inward oriented bending of the second portion of the outer skirt supported by the inflow prongs.

2. The prosthetic valve of claim 1, wherein the inflow prongs are configured to maintain an unbent configuration in a free state thereof.

3. The prosthetic valve of claim 1, wherein the inflow prongs are configured to bend radially outwards upon inflation of a balloon over which the prosthetic valve is crimped.

4. The prosthetic valve of claim 3, wherein the second portion of the outer skirt is configured to assume an outwardly flared configuration in the expanded state.

5. The prosthetic valve of claim 1, wherein the free ends of the inflow prongs define a distal-most end of the inflow prongs in the expanded state.

6. The prosthetic valve of claim 1, wherein the first portion of the outer skirt comprises a textured surface configured to encourage tissue growth thereover.

7. The prosthetic valve of claim 1, wherein the second portion of the outer skirt is devoid of textured surfaces that may encourage tissue growth thereover.

8. The prosthetic valve of claim 1, wherein the outer skirt extends between a first end and a second end; wherein the first end of the outer skirt is coupled to the frame between the inflow end and the outflow end of the frame; and wherein the second end of the outer skirt defines an inflow end of the prosthetic valve, which is distal to the inflow end of the frame in the expanded state.

9. The prosthetic valve of claim 1, wherein the second portion of the outer skirt is folded around the free ends of the inflow prongs.

10. The prosthetic valve of claim 9, wherein a fold of the outer skirt around the inflow prongs defines an inflow end of the prosthetic valve, which is distal to the inflow end of the frame in the expanded state.

11. The prosthetic valve of claim 1, further comprising a valvular structure mounted inside the frame and comprising a plurality of leaflets.

12. A prosthetic valve comprising:
a frame extending between an inflow end and an outflow end, and movable between a radially compressed state and a radially expanded state;
an outer skirt comprising a first portion disposed around the frame and a second portion extending distal to the inflow end of the frame; and
a reinforcing layer disposed over the second portion of the outer skirt;
wherein the reinforcing layer is configured to limit a radially-inward oriented bending of the second portion of the outer skirt; and
wherein the reinforcing layer is not disposed over the first portion of the outer skirt.

13. The prosthetic valve of claim 12, wherein a thickness of the second portion of the outer skirt, combined with the reinforcing layer, is greater than a thickness of the first portion of the outer skirt.

14. The prosthetic valve of claim 12, wherein an outer diameter of the prosthetic valve defined at a level of the second portion of the outer skirt is not greater than an outer diameter of the prosthetic valve defined at a level of the first portion of the outer skirt, in the radially compressed state.

15. The prosthetic valve of claim 12, wherein the reinforcing layer is stiffer than the outer skirt.

16. The prosthetic valve of claim 12, wherein the first portion of the outer skirt comprises a textured surface configured to encourage tissue growth thereover.

17. The prosthetic valve of claim 12, wherein the second portion of the outer skirt is devoid of textured surfaces that may encourage tissue growth thereover.

18. The prosthetic valve of claim 12, wherein the reinforcing layer comprises a thromboresistant surface.

19. The prosthetic valve of claim 12, wherein the outer skirt extends between a first end and a second end; wherein the first end of the outer skirt is coupled to the frame between the inflow end and the outflow end of the frame; and wherein the second end of the outer skirt is coupled to the inflow end of the frame.

20. The prosthetic valve of claim 12, wherein the second portion of the outer skirt is folded over itself.
